(19) United States
(12) Patent Application Publication
(10) Pub. No.: US 2007/0122462 A1
(43) Pub. Date: May 31, 2007

(54) Bandage with a hydrophilic foam containing silver

(75) Inventors: N. Satish Chandra, Landsdale, PA (US); Vinesh Naik, DuPont, PA (US); Anthony Michael Sosnowski, Stroudsburg, PA (US); Joel M. Furey, Stowe, VT (US); William F. McNally, Clarks Summit, PA (US)

Correspondence Address: AKERMAN SENTERFITT, P.O. BOX 3188, WEST PALM BEACH, FL, 33402-3188, US

(73) Assignee: Noble Fiber Technologies, LLC, Scranton, PA (US)

(21) Appl. No.: 11/432,876

(22) Filed: May 12, 2006

Related U.S. Application Data
(60) Provisional application No. 60/680,225, filed on May 12, 2005.

(51) Int. Cl. A61L 15/00; A61K 33/38

(52) U.S. Cl. 424/445; 424/618

(57) ABSTRACT

A compression stretch bandage formed from at least one layer of a stretchable, textile material forming a body of the bandage, a base material attached to the stretchable, textile material on a first side, and a silver material attached to the base material for reducing risk of infection. The bandage may be a flexible, stretchable, hydrophilic bandage that reduce the risk of infection at a wound by providing a moist environment that will aid in optimum release of silver ions into the wound.

### CROSS-REFERENCE TO RELATED APPLICATIONS

This application claims the benefit of U.S. Provisional Application No. 60/680,225 filed May 12, 2005.

### FIELD OF THE INVENTION

This invention is directed generally to an antibiotic bandages, and more particularly to wound dressing bandages that foster a moist wound-healing environment while minimizing the possibility of infection.

### BACKGROUND

Silver has been used as an antimicrobial since ancient times. Recent years have seen a renewed interest in silver as an antibiotic. This renewed interest is driven in part by the development of antibiotic-resistant bacteria, such as methicillin-resistant Staphylococcus aureus (MRSA). Resistant bacteria are especially problematic in wounds. Silver is a broad-spectrum antibiotic that is effective against such resistant bacteria and bacteria do not appear to develop resistance to silver. There is an urgent need for an antibiotic wound care product that uses silver to treat and/or prevent MSRA and other resistant infections.

Silver is also known to exhibit wound-healing properties. Expeditious wound healing benefits the patient in terms of increased comfort and decreased susceptibility to infection and secondary injury. There is a need for wound care products that utilize silver to increase the rate of wound healing.

Many presently existing antibiotic wound care products lose their antibiotic activity in a short period of time. This is especially true for wound care products that contain silver in an ionic form. Ionic silver is readily dissolved in an aqueous environment and dissipated. Such dressings must be replaced frequently often resulting in extreme pain or discomfort and inconvenience for the patient as the dressing is removed and a new dressing is applied. Similarly, silver creams (including silver sulfadiazine) must be consistently reapplied to the injured area, and the dressing must be removed for reapplication of the cream. There is a need for a wound care product that releases silver ions over an extended period of time and which alleviates the need for frequent removal or replacement of the dressing or application of silver creams.

Silver is commonly applied in as a silver salt. Such salts can be irritating to the skin. There is a need for a non-irritating silver wound care product that does not rely on silver salts for the delivery of silver ions. Moreover, prolonged contact with silver salts can cause argyria, which creates a pronounced, permanent ashen-gray skin discoloration that can be localized or universal. Thus, there is a need for a silver wound care product that does not cause argyria.

Silver is known to affect the operation of matrix metalloproteinases (MMPs). Excessive MMPs are known to interfere with wound healing. Excessive interference with MMPs can also interfere with wound healing. Presently existing silver-based wound care products often inhibit MMPs too much, thereby interfering with the wound healing process. Thus, there is a need for a silver wound care product that delivers an amount of silver, which limits the activity of MMPs without unduly restricting MMP activity.

### SUMMARY OF THE INVENTION

This invention is directed to a bandage formed from one or more layers of a stretchable, textile material forming a body of the bandage. The bandage may include a base material attached to the stretchable, textile material on a first side. In at least one embodiment, the base material may be a hydrophilic foam attached to the stretchable, textile material on a first side. A silver material may be attached to the hydrophilic foam for reducing risk of infection. The bandage may create a moist environment that is optimum for the release of silver ions into the wound bed to reduce the risk of infection in a wound on a patient.

The body of the bandage may include silver coated fibers. The silver coated fibers may be formed from silver coated nylon fibers. The base material may also include a silver material formed from a silver hydroxide or a silver powder, or both, that is attached to the base material for reducing risk of infection. The silver coated fibers, the silver hydroxide powder, or the silver powder may be used in amounts of about 0.5 percent and 10 percent by weight of the hydrophilic foam.

The silver material may be attached to a surface of the hydrophilic foam forming an antimicrobial layer on an outer surface of the bandage. In another embodiment, the silver material may be added to the base material during formation of the base layer. In such a configuration, the silver material may be positioned throughout the base material for increased antimicrobial effects.

An advantage of this invention is that the bandage may be formed from a silver metallized chopped fiber, a specialized metallic silver powder and silver hydroxide to provide ionic silver to treat and/or prevent MSRA and other resistant bacteria and fungi.

Another advantage of this invention is that the bandage uses ionic silver to inhibit the growth of bacteria that is detrimental to wound healing.

Yet another advantage of this invention is that the bandage includes ionic silver to increase the rate of wound healing and releases silver ions over a period of time. The release of silver ions is non-irritating and does not rely on silver salts for the delivery of silver ions.

Another advantage of this invention is that the bandage does not cause argyria in a patient to which the bandage is attached.

Still another advantage of this invention is that the bandage creates and maintains a moist wound-healing environment while preventing the growth of bacteria and fungi;

Another advantage of this invention is that the bandage maintains a moist environment, but eliminates unpleasant odors.

Yet another advantage of this invention is that the bandage uses metallic silver with a relatively large surface area that does not become detached from the base material.

Another advantage of this invention is that the bandage enables the delivery of an optimal dosage of silver ions.

Still another advantage of this invention is that the bandage delivers an amount of silver that limits the activity of MMPs without unduly restricting MMP activity.

Another advantage of this invention is that the bandage is easy and inexpensive to manufacture.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a perspective view of a bandage having aspects of this invention.

FIG. 2 is a cross-section of the bandage taken at line 2-2 in FIG. 1.

FIG. 3 is a perspective view of a sliver coated fiber.

FIG. 4 is a perspective view of an alternative bandage having aspects of this invention.

### DETAILED DESCRIPTION OF THE INVENTION

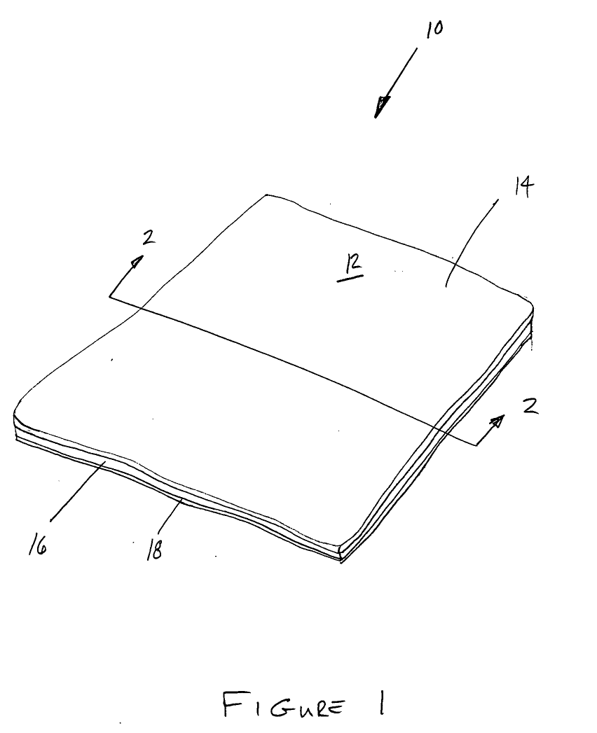

As shown in FIGS. 1-4, this invention is directed to a bandage 10 formed from one or more layers of a stretchable, textile material 12 forming a body 14 of the bandage 12. The bandage 10 may include a base material 16 attached to the stretchable, textile material on a first side. In at least one embodiment, the base material 16 may be a hydrophilic foam attached to the stretchable, textile material 12 on a first side. A silver material 18 may be attached to the hydrophilic foam for reducing risk of infection. The bandage 10, and specifically, the hydrophilic foam, may create a moist environment that is optimum for the release of silver ions into the wound bed to reduce the risk of infection in a wound on a patient.

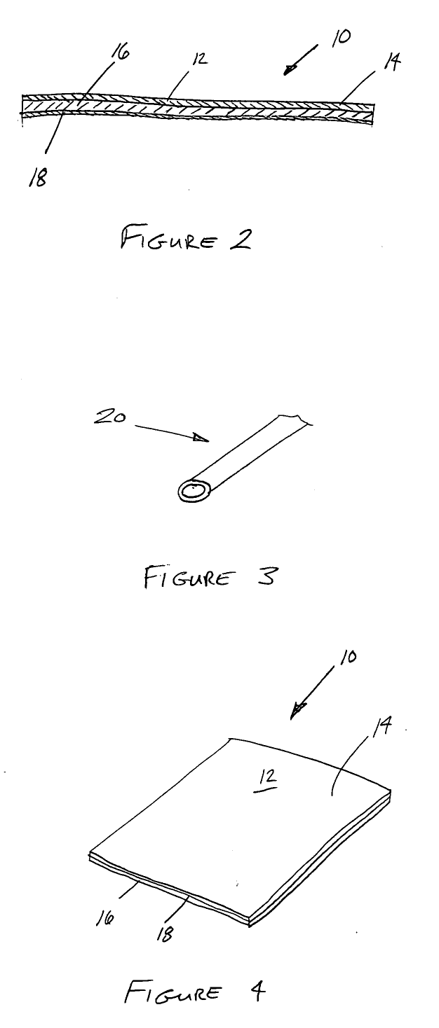

As shown in FIG. 2, the bandage 10 may be formed from a textile material 12. The textile material 12 may be a compression stretch bandage. The textile material may be any appropriate material. In at least one embodiment, the bandage 10 may include silver coated fibers 20, as shown in FIG. 3, attached to the textile material 12. The silver coated fibers 20 may be manufactured as described in U.S. Pat. No. 4,042,737, entitled “Process for producing crimped metal-coated filamentary materials, and yarns and fabrics obtained therefrom,” issued to Rohm and Haas Company (Philadelphia, Pa.), on Aug. 16, 1977, and are commercially available from Noble Fiber Technologies under the tradename X-STATIC. The silver coated fiber 20 may also be created in accordance with the process disclosed in U.S. patent application Ser. No. 10/666,568, the disclosure of which is hereby incorporated by reference.

The silver coated fiber 20 may be formed from a silver-coated nylon fiber. The following table describes characteristics of the silver coated fibers 20:

| | Length | Denier | Silver |
|---|---|---|---|
| | in Mils | (dpf) | (% w/w) |
| Outside range | 80-200 | .5-50 | 3-75% |
| Intermediate range | 40-150 | .7-30 | 9-60% |
| Optimal range | 30-100 | 1-10 | 12-30% |
| Ideal | ˜20 | ˜3 | ˜18 |

The silver material 18 may also be formed from a silver hydroxide. The silver hydroxide may be prepared as described in U.S. patent application Ser. No. 10/937,966, the disclosure of which is hereby incorporated by reference. In another embodiment, the silver material 18 may be a silver micro and nano powder. The silver micro and nano powder may be prepared as described in U.S. patent application Ser. No. 10/937,966. The above obtained powder may be surface modified if a need for a greater ion release exists for a particular application. This may be done as described in U.S. patent application Ser. No. 10/836,530, which is hereby incorporated by reference.

The percentage of the silver material 18 used together with the base material 16 may be controlled. In particular, the percentage of silver material 18 may be controlled by controlling the percentage of silver coated fibers 20 and silver powder added to the base material 16. In addition, the viscosity of the liquid inside the foam may be kept constant. The amount of silver material 18 used may also be controlled by altering the viscosity but keeping the percentage of silver coated fibers 20 or silver powders, or both the same.

The base material 16 may be a hydrophilic foam. The foam may be a chemically formed hydrophilic based polyurethane foam. The foam may have a thickness between about 0.1 inches and about 0.5 inches. The hydrophilic foam may be mixed with any of the three mentioned silver materials or any combination of the silver materials. The hydrophilic foam with silver coated fibers 20 can be prepared using materials in the following percentages:

| | Percent of X-Static by weight |
|---|---|
| Type of Silver Material | by volume |
| Chopped fiber (20 mil × 3 dpf) | 0.5-10 |
| Silver hydroxide powder | 0.5-10 |
| Silver powder with surface | 0.5-10 |
| conversion | |
| Silver powder w/o surface | 0.5-10 |
| conversion | |

Thus, the silver coated fibers 20 may be used in amounts of chopped fibers of between 0.5 percent and 10 percent by weight of the hydrophilic foam 16. The silver hydroxide powder may be used in amounts of about 0.5 percent and 10 percent by weight of the hydrophilic foam 16. The silver powder may be used in amounts of about 0.5 percent and 10 percent by weight of the hydrophilic foam 16. These materials create a flexible, stretchable and hydrophilic bandage 10. The moisture filled foam 16 with the silver material 18 provides an optimum moist environment which enables an optimum release of silver ions into a wound to increase a healing rate without significant scarring.

The silver material 18 may be attached to an outer surface of the base material 16, referred to as metallizing the base material 16, as shown in FIGS. 1-2, or may be incorporated in the base material 16 during formation of the base material 16, as shown in FIG. 4. The silver material 18 may be attached to the base material 16 via metallizing using, for instance, the process disclosed in U.S. patent application Ser. No. 11/209,567, which is incorporated by reference herein, whereby the silver material 18 forms a layer of silver on the base material 16. In another embodiment, the silver material 18 may be included in the base material 16 during formation of the base material 16. By including the silver material 18 in the base material 16, the silver material 18 may found throughout the base material 16 and not limited to being only on the surface of the base material.

The bandage 10 includes resistance at levels recordable in Ohms, which was an unexpected result. The foam 16 was conductive not only in the X and Y direction but also in the Z direction, which indicates that the foam 16 together with the silver material 18 have thermodynamic characteristics. It should be noted that lack of conductivity does not impair the ion release or the anti-microbial efficacy of the bandage 10.

### EXAMPLE

The base material 16 may be formed from a hydrophilic foam and a chopped fiber having about a 20 mil length and 3 dpf that was blown together. In another embodiment, the base material 16 may be formed from a hydrophilic foam and a silver hydroxide and silver powder, as noted below. The hydrophilic foam may have a dark brown color that darkens as time passes due to the hydroxide in the silver. However, the antimicrobial capabilities of the hydrophilic foam do not deteriorate with time. The following data was obtained from experiments:

| | | Ion | | | |
|---|---|---|---|---|---|
| | | release | | | |
| | | after 1 | | ASTM | ASTM |
| | % | hour (In- | Resistance | E-2149 | E-2140 |
| Foam | of X- | house | of sample in Z | against S. Aureaus | against |
| blown with | Static | protocol) | direction | (1 hr) | MRSA (1 hr) |
| Chopped | 4 | 9 | 1 × 10⁵ Ω | 99.9% | 99.9% |
| fiber (20 mil × 3 dpf) | | | | | |
| Chopped | 2 | 5 | 1 × 19⁵ Ω | 99.9% | 99.9% |
| fiber (20 mil × 3 dpf) | | | | | |
| Silver | 3 | 10 | 1 × 10⁸ Ω | 99.9% | 99.9% |
| hydroxide* | | | | | |
| Silver | 5 | 18 | 1 × 10⁷ Ω | 99.9% | 99.9% |
| hydroxide* | | | | | |
| Surface | 5 | 25 | 1 × 10⁵ Ω | 99.9% | 99.9% |
| converted silver | | | | | |
| X-Static powder | | | | | |
| Surface | 3 | 20 | 1 × 10⁵ Ω | 99.9% | 99.9% |
| converted silver | | | | | |
| X-Static | | | | | |

According to another test, the degree of hydrophilicity of the base material 16 was affected little by the addition of the silver material 18 that was added, as shown below:

Hydrophilicity Test (Based on Water retention Capacity)

| | Foam w/5% Ag | Foam w/out Ag |
|---|---|---|
| Orginal Length | 81 ± 2.0 mm | 81 ± 2.0 mm |
| Original Width | 60 ± 2.0 mm | 60 ± 2.0 mm |
| Original Thickness | 6.2 ± 0.8 mm | 6.2 ± 0.8 mm |
| H₂O Held | 16 ml | 16 ml |
| Wt. After mins. | | |
| 0 | 11.922 | 11.042 |
| 30 | 7.606 | 6.624 |
| 45 | 6.448 | 5.348 |
| 60 | 5.420 | 4.170 |
| 75 | 4.500 | 3.216 |
| 90 | 3.943 | 2.641 |
| 105 | 3.738 | 2.433 |

Oven Temp 100° C.
5.22% silver on the foam

[image]

While specific embodiments of the invention have been described in detail, it will be appreciated by those skilled in the art that various modifications and alternatives to those details could be developed in light of the overall teachings of the disclosure. Accordingly, the particular embodiments disclosed are meant to be illustrative only and not limiting as to the scope of the invention which is to be given the full breadth of the appended claims and any and all equivalents thereof.

We claim:

1. A bandage, comprising:
at least one layer of a stretchable, textile material forming a body of the bandage;
a base material attached to the stretchable, textile material on a first side; and
a silver material attached to the base material for reducing risk of infection.

2. The bandage of claim 1, wherein the at least one layer of a stretchable, textile material forming a body of the bandage includes silver coated fibers.

3. The bandage of claim 2, wherein the silver coated fibers are formed from silver coated nylon fibers.

4. The bandage of claim 1, wherein the silver material attached to the base material for reducing risk of infection comprises a silver hydroxide.

5. The bandage of claim 1, wherein the silver material attached to the base material for reducing risk of infection comprises a silver powder.

6. The bandage of claim 1, wherein the base material attached to the stretchable, textile material on a first side comprises a hydrophilic foam.

7. The bandage of claim 6, wherein the at least one layer of a stretchable, textile material forming a body of the bandage includes silver coated fibers.

8. The bandage of claim 7, wherein the silver coated fibers are formed from silver coated nylon fibers.

9. The bandage of claim 6, wherein the silver material attached to the base material for reducing risk of infection comprises a silver hydroxide.

10. The bandage of claim 6, wherein the silver material attached to the base material for reducing risk of infection comprises a silver powder.

11. The bandage of claim 1, wherein the silver material is included in the base material during formation of the base material such that the silver material is positioned through the base material.

12. A bandage, comprising:
at least one layer of a stretchable, textile material forming a body of the bandage;
a hydrophilic foam attached to the stretchable, textile material on a first side; and
a silver material attached to the hydrophilic foam for reducing risk of infection.

13. The bandage of claim 12, wherein the at least one layer of a stretchable, textile material forming a body of the bandage includes silver coated fibers.

14. The bandage of claim 13, wherein the silver coated fibers are formed from silver coated nylon fibers.

15. The bandage of claim 14, wherein the silver coated fibers are comprised of chopped fiber of between 0.5 percent and 10 percent by weight of the hydrophilic foam.

16. The bandage of claim 12, wherein the silver material attached to the base material for reducing risk of infection comprises a silver hydroxide powder.

17. The bandage of claim 16, wherein the silver hydroxide powder comprises 0.5 percent and 10 percent by weight of the hydrophilic foam.

18. The bandage of claim 12, wherein the silver material attached to the base material for reducing risk of infection comprises a silver powder.

19. The bandage of claim 18, wherein the silver powder comprises 0.5 percent and 10 percent by weight of the hydrophilic foam.

20. The bandage of claim 12, wherein the hydrophilic foam has a thickness of between about 0.1 inches and about 0.5 inches.
